(19) United States
(12) Patent Application Publication
(10) Pub. No.: US 2015/0223677 A1
(43) Pub. Date: Aug. 13, 2015

(54) ENDOSCOPE WITH LATERAL ILLUMINATION, USE AND METHOD

(71) Applicant: OLYMPUS WINTER & IBE GMBH, Hamburg (DE)

(72) Inventor: Peter SCHOUWINK, Hamburg (DE)

(73) Assignee: OLYMPUS WINTER & IBE GMBH, Hamburg (DE)

(21) Appl. No.: 14/695,526

(22) Filed: Apr. 24, 2015

Related U.S. Application Data
(63) Continuation of application No. PCT/EP2013/003004, filed on Oct. 7, 2013.

(30) Foreign Application Priority Data
Oct. 24, 2012 (DE) 102012219434.7

(51) Int. Cl. A61B 1/06; A61B 1/07

(52) U.S. Cl. CPC A61B 1/0623; A61B 1/07; A61B 1/0684; A61B 1/0638

(57) ABSTRACT

An endoscope with lateral illumination including: an endoscope shaft with an outer tube; an input lens arranged in a distal area of the outer tube, the input lens having a viewing direction pivotable in at least one pivot plane; two or more illumination optical-fiber bundles terminating at output surfaces at a distal tip, wherein a first illumination optical-fiber bundle generates an illumination, at a 0° viewing angle, and a second illumination optical-fiber bundle generates an illumination of another area with a lateral viewing angle or lateral viewing-angle range of between 0° and 90°; and one or more light-emitting diodes arranged in or on a circumference of the endoscope shaft in an area of the distal tip, wherein the light-emitting diode(s) is/are arranged in the pivot plane and/or around the pivot plane, in order to generate an illumination at a viewing angle or a viewing-angle range of about 90°.

### CROSS-REFERENCE TO RELATED APPLICATION

The present application is a continuation of PCT/EP2013/003004 filed on Oct. 7, 2013, which is based upon and claims the benefit to DE 10 2012 219 434.7 filed on Oct. 24, 2012, the entire contents of each of which are incorporated herein by reference.

### BACKGROUND

1. Field

The present application relates to an endoscope with lateral illumination, having an elongated endoscope shaft with an outer tube, in the distal area of which is arranged an input lens with a viewing direction pivotable in at least one pivot plane, wherein the endoscope shaft accommodates one or more illumination optical-fiber bundles, which terminate at output surfaces at a distal tip of the endoscope shaft, wherein at least a first illumination optical-fiber bundle generates an illumination, at a 0° viewing angle, of an area lying in front of the distal tip of the endoscope shaft, and at least a second illumination optical-fiber bundle generates an illumination of another area with a lateral viewing angle or lateral viewing-angle range of between 0° and 90°. The present application further relates to a use and a method for operating an endoscope.

According to the invention, it concerns an optical endoscope in which a lateral view is or can be set. Such endoscopes have either an optical system with an image sensor, which is arranged in the endoscope shaft or at the distal tip of the endoscope shaft, or rod lens relay sets, which relay the light captured at the distal tip to the proximal end of the endoscope to an ocular and if applicable to a video head.

A viewing direction change normally takes place through the pivoting of a prism behind a curved cover glass, which seals the distal end of the endoscope shaft.

When illumination through the endoscope shaft is required, the endoscope shaft also has, in addition to the optical system, means for illumination, i.e. for illumination of the area in front of the distal tip of the endoscope shaft. In some cases, it hereby concerns bundles of optical fibers, which receive light proximally from a light source, for example from light-emitting diodes (LEDs) or other light sources, and direct this light distally to the endoscope tip and emit it there for the illumination of the operative field.

Optical fibers in endoscope optics have a very large angle of radiation, but their beam directions should, whenever possible, all be directed to the center of the view field. Fibers deviating in the beam direction cause problematic differences in brightness in the view field. In order to seal and to secure the alignment given to them during assembly, the fibers are normally glued in the distal end area.

2. Prior Art

A corresponding endoscope optics with upper and lateral optical fiber bundles is known from DE 10 2004 023 024 A1. This concerns an optics with a lateral view, wherein a central window arranged asymmetrically in the endoscope shaft is provided to accommodate the received optics, and the asymmetrical edge areas are occupied by several optical fiber bundles, of which two lateral optical fiber bundles generate a lateral illumination at a viewing angle of approx. 45° and a central upper optical fiber bundle generates an illumination at a 0° angle.

However, a satisfactory solution for endoscopes with pivotable optics, which also have lateral viewing directions, up to for example 90°, has not yet been found. The object of the invention is thus to realise a sufficient illumination of an operative field even in endoscopes with adjustable viewing direction.

### SUMMARY

This object is solved by an endoscope with lateral illumination, having an elongated endoscope shaft with an outer tube, in the distal area of which is arranged an input lens with a viewing direction pivotable in at least one pivot plane, wherein the endoscope shaft accommodates one or more illumination optical-fiber bundles, which terminate at output surfaces at a distal tip of the endoscope shaft, wherein at least a first illumination optical-fiber bundle generates an illumination, at a 0° viewing angle, of an area lying in front of the distal tip of the endoscope shaft, and at least a second illumination optical-fiber bundle generates an illumination of another area with a lateral viewing angle or lateral viewing-angle range of between 0° and 90°, which is further developed in that one or more light-emitting diodes are arranged in or on the circumference of the endoscope shaft in the area of the distal tip, wherein the light-emitting diode or light-emitting diodes are arranged in the pivot plane and/or around the pivot plane, in order to generate an illumination at a viewing angle or a viewing-angle range about 90°.

Through the use of LEDs, the limiting factor that illumination optical-fiber bundles are bendable only to a limited degree is avoided. Within the available restricted volume within an endoscope shaft, it is hardly possible to bend illumination optical-fiber bundles by an angle of 90° without breaking at least some optical fibers of the bundle. The arrangement of LEDs on the circumference of the endoscope shaft in the area of the distal tip ensures in contrast a cost-effective, robust and sufficiently bright illumination of the lateral area, which is not illuminated or is insufficiently illuminated by the existing illumination optical-fiber bundles.

The areas illuminated by the second illumination optical-fiber bundles and by the light-emitting diode or the light-emitting diodes preferably overlap. A continuous illumination of the operative field is hereby achieved.

For the arrangement of the light-emitting diode or the light-emitting diodes, it is advantageous that the light-emitting diode or the light-emitting diodes is/are embedded in the outer tube or in a housing, which is arranged distally on the endoscope shaft. In the case of an embedding in the circumference either in the outer tube or in the corresponding housing on the distal tip, the LED or the LEDs do not protrude over the circumference of the endoscope shaft and thus do not form an obstacle. They can be protected for example by a transparent coat of paint or by a transparent film or a transparent tube.

Several light-emitting diodes are advantageously arranged symmetrically around the pivot plane. The outer light diodes are thereby advantageously switchable independently of a central light-emitting diode or several central light-emitting diodes. In this manner, it can be selected how large the area to be illuminated should be in the lateral direction. Thus, it is possible, for example in the case of a relatively small pivoting angle or respectively viewing angle, which only enters the area illuminated by the LEDs peripherally, to switch on only the central light-emitting diode or respectively central light-emitting diodes and, upon entering the area illuminated by the light-emitting diodes, also to switch on the outer light-emitting diodes in order to illuminate the area completely. The heat generation at the endoscope tip can thus be held low.

The light-emitting diode or light-emitting diodes is/are preferably designed as planar light-emitting diode or light-emitting diodes or several light-emitting diodes are arranged in a pattern spanning a surface. The goal is an even illumination of the operative area. Planar light-emitting diodes form for this a very homogeneous source, which can be achieved, in the case of small light-emitting diodes, through a corresponding distribution of the individual light-emitting diodes. Organic light-emitting diodes (OLEDs), which can be produced as planar light-emitting diodes, are particularly suitable.

Examinations, for example of different tissue types or clinical pictures, can advantageously be executed when one or more light-emitting diodes are designed to generate light with different colors or with adjustable colors. The colors can thereby be those in the visible range of the optical spectrum or even infrared or ultraviolet, wherein in this case correspondingly sensitive image sensors are used or these in connection e.g. with luminescence markers are used in the tissue of the patient.

A control apparatus is preferably included, by means of which the illumination by the illumination optical-fiber bundles and the light-emitting diode or light-emitting diodes is controllable depending on the set viewing angle, in order to illuminate the area lying in the direction of the set viewing angle in front of the distal tip of the endoscope shaft. In this manner, the selection of the illumination sources, i.e. the first illumination optical-fiber bundle, the second illumination optical-fiber bundle and light-emitting diode or light-emitting diodes, is no longer the job of the operator, but is rather selected automatically based on the set viewing angle.

The object underlying the invention is also solved through the use of a light-emitting diode or several light-emitting diodes in a previously described endoscope according to the invention for generating a lateral illumination. It is hereby possible to generate in a cost-effective and efficient manner a lateral illumination in the pivoting direction of the optics. Since only a lateral use of LEDs is necessary and light continues to occur in the straight-ahead direction and less strongly in the lateral direction by illumination optical-fiber bundles, the heat generation by the light-emitting diodes is limited to a great extent and does not lead to damage to the tissue or organs of a person to be examined or to be operated on.

Finally, the object underlying the invention is also solved through a method for operating a previously described endoscope according to the invention, wherein in the case of a pivoting of a viewing angle depending on the set viewing angle an illumination is generated via at least a first illumination optical-fiber bundle with 0° viewing direction, via at least a second illumination optical-fiber bundle with a lateral viewing angle or a lateral viewing-angle range of between 0° and 90° and/or via one or more light-emitting diodes, which are arranged in or on the circumference of the endoscope shaft in the area of the distal tip, wherein the light-emitting diode or light-emitting diodes are arranged in the pivot plane and/or around the pivot plane, in order to generate an illumination at a viewing angle or a viewing-angle range about 90°.

The advantages, properties and characteristics named for the individual subject matters of the invention, i.e. the endoscope, the use and the method, also apply respectively to the other subject matters of the invention, which relate to each other.

Further characteristics of the invention will become apparent from the description of embodiments according to the invention together with the claims and the included drawings. Embodiments according to the invention can fulfill individual characteristics or a combination of several characteristics.

### BRIEF DESCRIPTION OF THE DRAWINGS

The invention is described below, without restricting the general idea of the invention, using exemplary embodiments with reference to the drawings, whereby we expressly refer to the drawings with regard to all details according to the invention that are not explained in greater detail in the text. The figures show in:

FIG. 1 is a schematic cross-sectional representation through an endoscope shaft of an endoscope according to the invention; and

FIG. 2 is a schematic side view of an area of the distal tip of an endoscope shaft of an endoscope according to the invention.

### DETAILED DESCRIPTION

In the drawings, the same or similar types of elements and/or parts are provided with the same reference numbers so that a re-introduction is omitted.

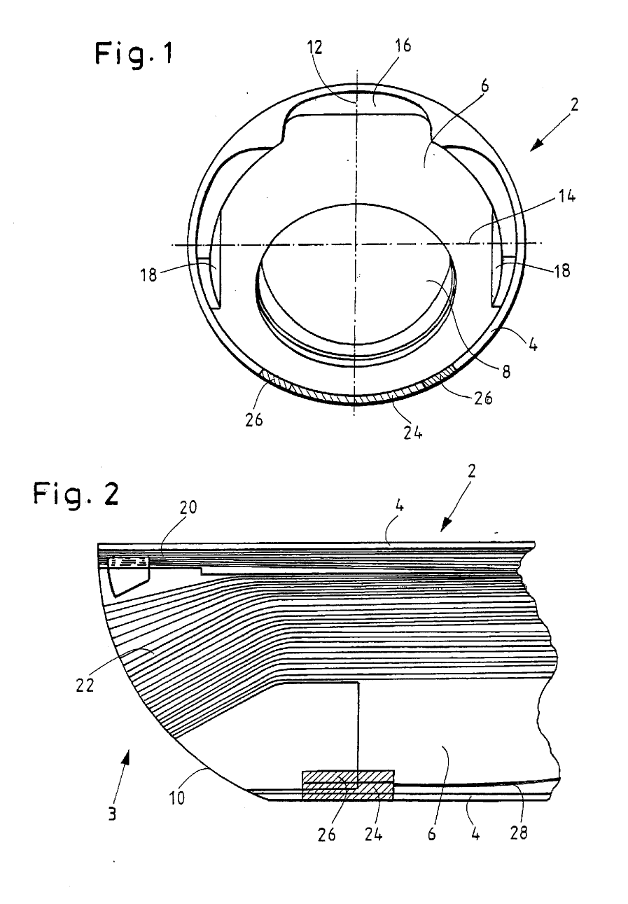

FIG. 1 shows schematically in cross-section an endoscope shaft 2 of an endoscope according to the invention in the area of the distal tip 3. A fiber tube 6, which has on one side a window opening 8 for an optical assembly and on the other side an upper outlet opening 16 and lateral outlet openings 18 for illumination optical-fiber bundles, is arranged within an outer tube 4. The diameter of the optical assembly, which is set in the window opening 8, is smaller than the diameter of the endoscope shaft 2 and is set in offset with respect to the edge so that there is space around the window opening 8 for the outlet opening 16, 18 for the illumination of an operative field in front of the distal tip of the endoscope.

The optical assembly, which is to be inserted into the window opening 8, can be a rigidly laterally looking assembly or an assembly with pivotable viewing direction, for example by means of a pivot prism arrangement, wherein the pivot plane, i.e. the plane, in which the pivoting of the viewing direction or respectively of the prism takes place, is labeled with reference number 12. A horizontal axis 14, which also runs parallel to a rotational axis of a corresponding pivot prism arrangement, is shown perpendicular to it, also transversally to the longitudinal extension of the endoscope shaft 2.

Below the window opening 8, a number of light-emitting diodes 24, 26, which are aligned such that they illuminate a lower area, which is arranged partially in front of the distal tip of the endoscope shaft 2, is shown in the area of the outer tube 4. These are a wide light-emitting diode 24, which is arranged symmetrically around the pivot plane 12, as well as two lateral light-emitting diodes 26, which are arranged symmetrically around the pivot plane 12 of the first light-emitting diode 24. These can be planar light-emitting diodes or light-emitting diode fields with individual light-emitting diodes, which span the corresponding surfaces.

FIG. 2 shows laterally the area of the distal tip 3 of the endoscope shaft 2 according to FIG. 1, wherein in this case the illumination optical-fiber bundles 20, 22, which are arranged behind each other in the viewing plane, are additionally shown. A first illumination optical-fiber bundle 20, which is aligned in a 0° viewing direction and radiates, is thereby arranged in the upper part, according to the upper outlet opening 16 from FIG. 1. The lateral illumination optical-fiber bundle 22 is arranged in the area of the distal tip 3 at an angle, in the present case approx. 30°, deviating from the 0° viewing direction, and illuminates an area lying more laterally in the pivot direction. At the distal tip 3, the endoscope shaft 2 is closed by a curved cover glass 10.

In the lower area of the endoscope shaft 2, the light-emitting diodes 24 and lateral light-emitting diodes 26 from FIG. 1 are shown from the side. These have comparatively wide illumination characteristics and serve to illuminate the area in front of and under the endoscope tip 3, which cannot be achieved by the light from the optical fibers of the second illumination optical-fiber bundles 22. Furthermore, a power supply line 28 is shown, which supplies the light-emitting diodes 24, 26 with power proximally.

By means of further illumination by the light-emitting diodes 24, 26, it is now possible to set a very lateral viewing angle of up to 90° and to thereby have a fully illuminated operative field. The arrangement of the light-emitting diodes 24, 26 is thereby selected such that the 90° viewing angle of the optics remains free.

All named characteristics, including those taken from the drawings alone and individual characteristics, which are disclosed in combination with other characteristics, are considered alone and in combination as essential for the invention. Embodiments according to the invention can be realised by individual features, or a combination of several features.

### LIST OF REFERENCE NUMBERS

2 Endoscope shaft
3 Distal tip
4 Outer tube
6 fiber tube
8 Window opening
10 Cover glass
12 Pivot plane
14 Horizontal axis
16 Upper outlet opening
18 Lateral outlet opening
20 First illumination optical-fiber bundle
22 Second illumination optical-fiber bundle
24 Light-emitting diode
26 Lateral light-emitting diode
28 Power supply line

What is claimed is:

1. An endoscope with lateral illumination comprising:
an elongated endoscope shaft with an outer tube;
an input lens arranged in a distal area of the outer tube, the input lens having a viewing direction pivotable in at least one pivot plane;
two or more illumination optical-fiber bundles accommodated in the endoscope shaft, the two or more illumination optical-fiber bundles terminating at output surfaces at a distal tip of the endoscope shaft, wherein at least a first illumination optical-fiber bundle generates an illumination, at a 0° viewing angle, of an area lying in front of the distal tip of the endoscope shaft, and at least a second illumination optical-fiber bundle generates an illumination of another area with a lateral viewing angle or lateral viewing-angle range of between 0° and 90°; and
one or more light-emitting diodes arranged in or on a circumference of the endoscope shaft in an area of the distal tip, wherein the one or more light-emitting diodes are arranged in the pivot plane and/or around the pivot plane, in order to generate an illumination at a viewing angle or a viewing-angle range of about 90°.

2. The endoscope according to claim 1, wherein the areas illuminated by the second illumination optical-fiber bundle and by the one or more the light-emitting diodes overlap.

3. The endoscope according to claim 1, wherein the one or more light-emitting diodes are embedded in the outer tube or in a housing, which is arranged distally on the endoscope shaft.

4. The endoscope according to claim 1, wherein the one or more light-emitting diodes comprise a plurality of light-emitting diodes arranged symmetrically around the pivot plane.

5. The endoscope according to claim 4, wherein outer light-emitting diodes of the plurality of light-emitting diodes are switchable independently of one or more central light-emitting diodes of the plurality of light-emitting diodes.

6. The endoscope according to claim 1, wherein the one or more light-emitting diodes are configured as one or more planar light-emitting diode diodes or a plurality of light-emitting diodes arranged in a pattern spanning a surface.

7. The endoscope according to claim 1, wherein the one or more light-emitting diodes are configured to generate light with different colors or with adjustable colors.

8. An endoscope according to claim 1, further comprising a control apparatus for controlling the illumination by the two or more illumination optical-fiber bundles and the one or more light-emitting diodes depending on the viewing angle, in order to illuminate the area lying in the direction of the viewing angle in front of the distal tip of the endoscope shaft.
